(19) United States
(12) Patent Application Publication
(10) Pub. No.: US 2024/0355415 A1
(43) Pub. Date: Oct. 24, 2024

(54) METHODS AND DEVICES FOR NON-INVASIVE PRENATAL TESTING

(71) Applicants: INSTITUT NATIONAL DE LA SANTE ET DE LA RECHERCHE MEDICALE, Paris (FR); CENTRE NATIONAL DE LA RECHERCHE SCIENTIFIQUE (C.N.R.S.), Paris (FR); UNIVERSITE CÔTE D'AZUR, Nice (FR); CHU DE NICE, Nice (FR)

(72) Inventors: Silvia BOTTINI, Nice (FR); David PRATELLA, Nice (FR); Marco MILANESIO, Nice (FR); Véronique DUBOC, Nice Cedex 3 (FR); Véronique PAQUIS-FLUCKLINGER, Nice Cedex 3 (FR)

(21) Appl. No.: 18/688,211

(22) PCT Filed: Sep. 3, 2021
(86) PCT No.: PCT/IB2021/000635

§ 371 Date: Feb. 29, 2024

(51) Int. Cl. G16B 20/10; C12Q 1/6827; G16B 40/20; G16H 10/40; G16H 50/20

(52) U.S. Cl. CPC G16B 20/10; C12Q 1/6827; G16B 40/20; G16H 10/40; G16H 50/20

(57) ABSTRACT

Here, a suite of methods and devices is provided, allowing the implementation of a strategy for Non-Invasive Prenatal Testing (NIPT) results validation in clinical practice: a package to perform NIPT analysis, a semi-supervised approach to create synthetic sequences and to estimate confidence intervals for aneuploidies prediction and a package to test the reliability of NIPT results based on confidence intervals. These new tools were validated on 2 cohorts including confirmed ancuploidies, demonstrating sensitivity and specificity of 100%. Thus, the invention relates to a method for assessing the fetal fraction (ff) and sequencing depth (sd) in NIPT; devices for implementing the method, computer program products and computer readable mediums. This result yields the conclusion that thresholds of ff, sd and E value need to be assessed for each data analysis pipeline, chromosome and cohort. The provided devices and methods are thus of wide interest as it allows to identify these thresholds in a laboratory—specific fashion to improve NIPT performances.

### FIELD OF THE DISCLOSURE

The invention relates to the field of non-invasive prenatal testing.

In particular, the invention relates to the detection of fetal chromosomal abnormalities, and more particularly of fetal ancuploidies.

### BACKGROUND OF THE DISCLOSURE

The development of non-invasive prenatal testing (NIPT) has revolutionized the landscape of prenatal diagnosis by offering to pregnant women a safe alternative to test for fetal aneuploidies such a trisomy 21 (Down syndrome). This test is based on circulating cell free DNA fragments (cfDNA) in maternal blood. Maternal plasma contains both maternal and fetal cfDNAs that are analyzed by low coverage next generation sequencing (NGS). The fetal fraction (ff) is the percentage of cfDNA in maternal plasma of fetoplacental origin (ff=fetal cfDNA/fetal cfDNA+maternal cfDNA). It increases during pregnancy with an average of 10 to 15% between 10 to 20 gestational weeks as assessed by direct measurement of fetal cfDNA in maternal blood. The ff depends on different maternal parameters including body mass index (BMI) and diseases as well as on fetoplacental parameters. Reliability of NIPT is high but depends on a sufficient ff.

It has been proposed that the ff should be at least 4% to validate NIPT results according to Palomaki et al. («DNA sequencing of maternal plasma to detect Down syndrome: an international clinical validation study. Genet Med. 2011; 13(11):913-920).

Several bioinformatics tools have been developed for ff estimation with very different performances, as reported in Peng & Jiang (“Bioinformatics Approaches for Fetal DNA Fraction Estimation in Non-Invasive Prenatal Testing”. International Journal of Molecular Sciences. 2017; 18(2):453), Beek et al. (“Comparing methods for fetal fraction determination and quality control of NIPT samples”. Prenatal Diagnosis. 2017; 37(8):769-773) and Hestand et al. («Fetal fraction evaluation in non-invasive prenatal screening (NIPS)”. Eur J Hum Genet. 2019; 27(2):198-202).

Early approaches to calculate the ff are based on the count of reads observed of the Y chromosome, as reported by Hudecova et al. (“Maternal Plasma Fetal DNA Fractions in Pregnancies with Low and High Risks for Fetal Chromosomal Aneuploidies”. PLoS One. 2014; 9(2)), including the DEFRAG algorithm.

Although being the most accurate, these methods are informative for male fetuses only. Two other approaches, independent of the sex of the fetus, have been developed: Seqff and Sanefalcon, as reported by Kim et al. (“Determination of fetal DNA fraction from the plasma of pregnant women using sequence read counts. Prenatal Diagnosis. 2015; 35(8):810-815) and Straver et al. (“Calculating the fetal fraction for Non-Invasive Prenatal Testing based on genome-wide nucleosome profiles”. Prenat Diagn. 2016; 36(7):614-621). The first one is based on the assumption that fetal and maternal fragments are not evenly distributed on the genome; thus authors used a large cohort to pre-train a model able to detect these small differences within a predefined bin resolution and to estimate the ff. The second is founded on the hypothesis that a differential nucleosome packaging exists between fetal and maternal DNA leading to a population of shorter fetal fragments. Thus the authors proposed to estimate the ff by exploiting the spatial distribution of reads on the estimated nucleosome profiles. In addition to the absence of a gold standard method to calculate the ff value, there is no universal ff threshold applicable across diagnostics laboratories.

There is thus a need:
to provide more reliable and sensitive methods and devices for non-invasive prenatal testing, and, moreover, to assess the reliability of pre-existing methods and devices in NIPT,
to improve test reliability in case of low fetal fraction (ff) and/or low sequencing depth (sd). A crucial aspect consists in reducing the number of false negatives, which may lead to undiagnosed aneuploidies, and false positives, which causes unnecessary invasive testing,
for methods and devices which are predictive for a broader selection of individuals, and for a broader selection of potential disorders during pregnancy, and
for methods for the diagnosis of such conditions, which remain cost-effective and less invasive or compromising during pregnancy.

The invention has for purpose to meet the above-mentioned needs.

### SUMMARY

According to a first main embodiment, the invention relates to a method for assessing the fetal fraction (ff) and sequencing depth (sd) in Non-Invasive Prenatal Testing (NIPT), comprising the steps of:
a) providing a set of sequences reads from a maternal biological sample, wherein for each sequence read the fragment length is known,
b) assigning a weight factor to one or more fragment length(s) of the set of sequences reads,
c) computing a synthetic profile, whereby a selection of at least one sequence read from the maternal biological sample is removed, or replaced with a selection of sequence read(s) from non-pregnant sample(s),
d) computing a value E corresponding to the synthetic profile, based on at least one of (i) the total number of reads with fragment length in a reference fetal range and (ii) the number of reads with fragment length in a chromosome of interest (T) in a reference fetal range, and
e) estimating the fetal fraction and sequencing depth of the synthetic profile based at least on said value E.

In a second main embodiment, the invention relates to a method for determining the reliability of Non-Invasive Prenatal Testing (NIPT), using a decision tree trained beforehand on reference profiles, comprising the steps of:
a) providing, from a maternal biological sample, a fetal fraction (ff), a sequencing depth (sd), a synthetic profile and a value E corresponding to said synthetic profile, according to any of the preceding claims,
b) calculating a Z-score for said synthetic profile by comparing it to Z-scores of said reference profiles,
c) feeding said decision tree with said calculated Z-score and said fetal fraction, sequencing depth and value E of said synthetic profile, in order to classify said synthetic profile in a group by comparing said calculated Z-score to a Z-score threshold value, and
d) determining, from said classification, a reliability score (Rscore) for a NIPT of said maternal biological sample.

The invention also relates to a device for implementing the method for assessing the fetal fraction (ff) and sequencing depth (sd) in Non-Invasive Prenatal Testing (NIPT) according to the invention.

In a preferred embodiment, said device comprises a decision tree trained beforehand on reference profiles, the device being configured to implement the method for determining the reliability of Non-Invasive Prenatal Testing (NIPT) according to the invention.

### DESCRIPTION OF THE FIGURES

FIG. 1. In FIG. 1A, NiPTUNE (the complete suite) is composed of seven blocks, each comprising one or multiple modules. The corresponding modules are indicated in white boxes. The name of the associated python script is reported in italic. The last column reports the outcome of each block. In FIG. 1B. iSanefalcon (module for ff estimation) is composed of five main steps, each containing one or multiple modules. The corresponding modules are indicated in white boxes. In the last column is reported the result of each step.

FIG. 2. GenomeMixer: a novel bioinformatic tool to create synthetic sequencing of pregnant women. Read length distributions of euploid samples from pregnant women (SPW) and non-pregnant women (SNPW) from cohort 1 (FIG. 2A) and cohort 2 (FIG. 2B). Distributions are colored according to the ff estimated by Seqff for the corresponding sample. A gradient of shades of one color is used to represent the range of ff for each cohort. SNPW were added as control. In FIG. 2C GenomeMixer workflow. Main steps of GenomeMixer are reported in the first column. Cartoons depict how samples are generated by GenomeMixer_sd or GenomeMixer_ff, respectively. Both take as input SPW with trisomy, and GenomeMixer_ff uses SNPW as well. Reads are labeled, using length-dependent weights, as most likely belonging to maternal or fetal population. n reads are then sampled, where n depends on the percentage of reads chosen by the user. Finally, GenomeMixer_sd removes the sampled reads, while GenomeMixer_ff replaces them with reads sampled among SNPW reads. The procedure is iterated depleting or replacing increments of a fixed percentage of reads from the initial read count until all reads are either removed or substituted. Color code: black bars, SPW reads before labeling; violet bars, SNPW reads; green bars, reads labeled as fetal reads; red bars, reads labeled as maternal reads.

FIG. 3. The impact of ff and sd on fetal chromosomal aberration prediction. In FIG. 3A Samples generated with GenomeMixer_ff, upper panel (A-F) and in FIG. 3B GenomeMixer_sd lower panel, (G-L). Starting from 30 native aneuploid (NA) samples, we generated 19 synthetic aneuploid (SA) samples per NA by replacing increments of 5% from the initial reads counts. NA starting pools comprise either male fetuses only for Defrag a (A-E, G-K) or all NA for Seqff (B-F,H-L). Trends of the modulated parameters (ff: A-B, sd: G-H) during generation of synthetic samples are shown. Trends of parameters to keep stable (sd: C-D, ff: I-J) along iterations are shown. Relationships between modulated parameters (ff: E-F, sd: K-L) during generation of samples and the Z-score are shown. Samples with Z-score below 5 are colored in red. NA samples are represented as squares, SA as triangles.

FIG. 4. E values of chr 18 or chr 21 for each sample as function of Z-scores for both cohorts. Panels A and B both relatye to cohort 1, respectively for chromosome 18 and 21. Panels C and DB both relatye to cohort 2, respectively for chromosome 18 and 21.

FIG. 5. Assessment of confidence intervals for reliable NIPT for clinical practice with ff estimated by Seqff. In FIG. 5A: Decision trees showing the confidence intervals for the three parameters: E value, sd and ff calculated with Seqff. Each node represents a discriminant value for one of the parameters (sd: circle, ff: rectangle, E value: smoothed rectangle). Rscore is reported for each confidence interval at the bottom of the tree. Percentage of SA by Rscore, generated with either (FIG. 5B) GenomeMixer_ff or (FIG. 5C) GenomeMixer_sd for each % of replaced or removed reads.

FIG. 6. Top histograms are showing counts of samples for which ff could be determined. Percentage of samples by Rscore for each category (NE18, NE21, NA18, NA21, SA18, SA21). FIG. 6A corresponds to a Rscore equal or superior to 0.9 which corresponds to “highly reliable”; FIG. 6B corresponds to a Rscore equal or superior to 0.2 and inferior to 0.8 which corresponds to “reliable”; FIG. 6C corresponds to a Rscore inferior to 0.2 which corresponds to “not reliable”.

FIG. 7. Assessment of confidence intervals for reliable NIPT in clinical practice for ff estimated by Defrag a. A) Decision trees showing the confidence intervals for the three parameters: E value, sd and ff calculated with Defrag a. Each node represents a discriminant value for one of the parameters (sd: circle, ff: rectangle, E value: smoothed rectangle). Rscore is reported for each confidence interval at the bottom of the tree. Percentage of SA by Rscore, generated with either B) GenomeMixer_ff or C) GenomeMixer_sd for each % of replaced or removed reads. Top histograms show the count of samples for which ff could be determined. D) Percentage of samples by Rscore for each category (NE18, NE21, NA18, NA21, SA18, SA21) are provided as in FIG. 6.

### DETAILED DESCRIPTION OF THE DISCLOSURE

Here, a suite of methods and devices is provided, allowing the implementation of a strategy for NIPT results validation in clinical practice: NiPTUNE, a package to perform NIPT analysis, GenomeMixer a semi-supervised approach to create synthetic sequences and to estimate confidence intervals for aneuploidies prediction and TRUST to test the reliability of NIPT results based on confidence intervals. These new tools were validated on 2 cohorts including a total of 1439 samples with 31 confirmed aneuploidies demonstrating sensitivity and specificity of 100%.

In particular, a new indicator “E value” or “value E”, is reported, which is based on at least one of (i) the total number of reads with fragment length in a reference fetal range and (ii) the number of reads with fragment length in a chromosome of interest (T) in a reference fetal range.

To the best of their knowledge, this is the first study of the relationship between fetal fraction (ff), sequencing depth (sd), and Z-score showing that they are profoundly connected to the said E value. Importantly, it is shown herein that single thresholds for ff, sd and E value do not suffice to achieve reliable NIPT but more complex entangled threshold are needed to stratify tests. Furthermore, it is shown that, depending on the device/method used to calculate ff, different thresholds and intervals are obtained. This result yields the conclusion that thresholds of ff, sd and E value need to be assessed for each data analysis pipeline, chromosome and cohort. The provided devices and methods are thus of wide interest as it allows to identify these thresholds in a laboratory-specific fashion to improve NIPT performances.

### Method for Assessing the Fetal Fraction (ff) and Sequencing Depth (sd) in NIPT

The inventors now provide herein a method for assessing the fetal fraction (ff) and sequencing depth (sd) in Non-Invasive Prenatal Testing (NIPT), comprising the steps of:
a) providing a set of sequences reads from a maternal biological sample, wherein for each sequence read the fragment length is known,
b) assigning a weight factor to one or more fragment length(s) of the set of sequences reads,
c) computing a synthetic profile, whereby a selection of at least one sequence read from the maternal biological sample is removed, or replaced with a selection of sequence read(s) from non-pregnant sample(s),
d) computing a value E corresponding to the synthetic profile, based on at least one of (i) the total number of reads with fragment length in a reference fetal range and (ii) the number of reads with fragment length in a chromosome of interest (T) in a reference fetal range, and
e) estimating the fetal fraction and sequencing depth of the synthetic profile based at least on said value E.

According to one alternative embodiment, the method is for assessing the fetal fraction (ff) in Non-Invasive Prenatal Testing (NIPT).

According to one alternative embodiment, the method is for assessing the sequencing depth (sd) in Non-Invasive Prenatal Testing (NIPT).

In one exemplary embodiment, the method further comprises a step of isolating cell-free DNA (cfDNA) from the maternal biological sample.

In one exemplary embodiment, the methods reported herein, may comprise a step of sequencing isolated cell-free DNA (cfDNA) from the maternal biological sample, thereby obtaining a set of sequence reads.

In one exemplary embodiment, the methods reported herein, may comprise a step of amplifying isolated cell-free DNA (cfDNA) from the maternal biological sample.

In one exemplary embodiment, the methods reported herein, may comprise a step of amplifying isolated cell-free DNA (cfDNA), and sequencing the amplified cfDNA, thereby obtaining a set of sequence reads.

In one exemplary embodiment, the maternal biological sample is a biological sample selected from a blood sample or fraction thereof, and more particularly is a maternal plasma sample.

In one exemplary embodiment, the method further comprises a step of isolating cell-free DNA (cfDNA) from the maternal biological sample.

In one exemplary embodiment, the method comprises assigning a weight factor to a plurality of fragment lengths of the set of sequence reads.

In one exemplary embodiment, the method comprises a step of computing a synthetic profile, whereby a selection of a plurality of sequence reads from the maternal biological sample is removed or replaced with a selection of a plurality of sequence reads from non-pregnant sample(s).

In one exemplary embodiment, the method comprises a step of removing at least one read from the sequences reads, thereby generating a synthetic profile with a modulated sequencing depth (sd) compared to the synthetic profile of step c).

In one exemplary embodiment, the method further comprises a step of replacing at least one read from the sequences reads, thereby generating a synthetic profile with a modulated fetal fraction (ff) compared to the synthetic profile of step c).

In a preferred embodiment, the value E is defined as:

$${E}_{ChrT} = \frac{ff * {n}_{( fetal \_ range )}^{chrT} - {n}_{( fetal \_ range )}^{genome} * ff * {p}_{chrT}}{\sqrt{{n}_{( fetal \_ range )}^{genome} * ff * {p}_{chrT} ( 1 - ff * {p}_{chrT} )}}$$

Where n_{(fetral_range)}^{genome} corresponds to the total number of reads with fragment length in a reference fetal range,
Where n_{(fetal_range)}^{chrT} corresponds to the number of reads with fragment length in the chromosome of interest T in a reference fetal range,
Where

$${p}_{chrT} = \frac{n reads chrT}{n reads genome}$$

corresponds to the number of reads on the chromosome of interest T divided by the total number of reads in the synthetic profile.

Advantageously, the more the value E deviates from 0 the likelier the chromosome of interest T is to present an anomaly.

The value E may be a number greater than 0.

In one exemplary embodiment, the chromosome of interest T may be selected from any of the human or non-human chromosomes, or a plurality thereof, preferably human chromosomes, including those selected from the list consisting of chromosome(s) 1, 2, 3, 4, 5, 6, 7, 8, 9, 10, 11, 12, 13, 14, 15, 16, 17, 18, 19, 20, 21, 22, and either one of X and/or Y chromosomes.

According to some embodiments, the chromosome of interest T is selected from human sex chromosomes.

According to some embodiments, the chromosome of interest T is selected from one or more chromosome(s) which is/are not human sex chromosomes.

In one exemplary embodiment, the chromosome of interest T is selected from the group consisting of one or more chromosome(s) selected from the group consisting of: 13, 18, 21, X and Y.

In one exemplary embodiment, the chromosome of interest T is selected from the group consisting of one or more chromosome(s) selected from the group consisting of: 13, 18 and 21; in particular chromosome 18 and/or chromosome 21.

In one exemplary embodiment, the method further comprises a step of determining fetal gender from the maternal biological sample.

In particular, the method may further comprise a step of comparing said proportion of reads to a reference value, thereby determining fetal gender from the maternal biological sample.

### Method for Determining the Reliability of Non-Invasive Prenatal Testing

The inventors also provide herein a method for determining the reliability of Non-Invasive Prenatal Testing (NIPT), using a decision tree trained beforehand on reference profiles, comprising the steps of:
a) providing, from a maternal biological sample, a fetal fraction (ff), a sequencing depth (sd), a synthetic profile and a value E corresponding to said synthetic profile, according to the method for assessing the ff and sd of the present disclosure,
b) calculating a Z-score for said synthetic profile by comparing it to Z-scores of said reference profiles,
c) feeding said decision tree with said calculated Z-score and said fetal fraction, sequencing depth and value E of said synthetic profile, in order to classify said synthetic profile in a group by comparing said calculated Z-score to a Z-score threshold value, and
d) determining, from said classification, a reliability score (Rscore) for a NIPT of said maternal biological sample.

In a preferred embodiment, at step c), said synthetic profile is classified in a group of aneuploid profiles or a group of euploid profiles, so as to determine the reliability of Non-Invasive Prenatal Testing in detecting fetal aneuploidy.

In one exemplary embodiment, the fetal aneuploidy is a human fetal aneuploidy of a chromosome of interest (T), in particular those selected from the group consisting of chromosomes 13, 18, 21, X and Y; preferably those selected from the group consisting of chromosomes 13, 18, and 21.

The use of a decision tree allows to obtain different intervals for the samples corresponding to different levels of reliability, represented by said R-score, based on the fetal fraction, the sequencing depth and the value E.

The Z-score threshold value may be determined, for instance, by the Wisecondor X program, with default values, as reported in Raman et al. (“WisecondorX: improved copy number detection for routine shallow whole-genome sequencing”. Nucleic Acids Research. 2019. Vol. 47, No. 4, 1605-1614).

The reliability score is preferably a probability, comprised between 0 and 1. Preferably, the closest the reliability score is to 1, the more the NIPT of the tested sample is reliable.

According to some embodiments, aneuploid samples from the two cohorts and synthetic aneuploid samples generated with GenomeMixer were used to calculate minimal thresholds for sd, ff and value E to obtain a reliable NIPT. We used a decision tree approach using the R package caret (https://cran.r-project.org/web/packages/caret/index.html), specifically the function rpart. Briefly, we used WisecondorX to calculate the Z-score of synthetic samples and Seqff and Defrag_a to assess their ff.

The sd and the values E were calculated using the modules despina.py and nereid.py from the NiPTUNE pipeline. A threshold of 5 on the Z-score was used to classify samples as “Aneuploid” (Z-score>=5) and “Euploid” (Z-score<5).

This threshold is defined as the default one by the tool WisecondorX. Then, we fed the decision tree with the values of sd, ff, value E and the classification to obtain a decision tree that groups samples. Two trees were calculated, one for Seqff and one for Defrag_a.

The features defined above for the method for assessing the fetal fraction and sequencing depth in Non-Invasive Prenatal Testing apply to the method for determining the reliability of Non-Invasive Prenatal Testing, and vice and versa.

### Device(s)

In yet another aspect, the invention relates to a device for implementing the method for assessing the fetal fraction (ff) and sequencing depth (sd) in Non-Invasive Prenatal Testing (NIPT) according to the invention.

In a preferred embodiment, the device comprises a decision tree trained beforehand on reference profiles, the device being configured to implement the method for determining the reliability of Non-Invasive Prenatal Testing (NIPT) according to the invention.

In another embodiment, the invention relates to a device for implementing the method for determining the reliability of Non-Invasive Prenatal Testing (NIPT) according to the invention, said device comprising a decision tree trained beforehand on reference profiles, the device being configured to implement the method for determining the reliability of Non-Invasive Prenatal Testing (NIPT) according to the invention.

The features defined above for the methods apply to the devices.

Said fetal fractions, sequencing depths, E values, Z-score(s), reliability scores and Z-score threshold values used in the methods according to the invention may be transmitted to a user by any suitable mean, for example by being displayed on a screen of an electronic device, printed, or by vocal synthesis.

Each step of the methods according to the invention may be carried out on one or more electronic systems, in particular a personal computer, a calculation server or a medical imaging device, preferably comprising at least a microcontroller and a memory.

### Computer Program Products

Such methods according to the invention are advantageously performed by means of computer programs, automatically on any electronic system comprising a processor, especially a computer.

In yet another aspect, the invention relates to a computer program product comprising a support and stored on this support instructions that can be read by a processor, these instructions being configured to assess the fetal fraction (ff) and sequencing depth (sd) in Non-Invasive Prenatal Testing (NIPT) according to the invention, and/or to determine the reliability of Non-Invasive Prenatal Testing (NIPT) according to the invention.

The invention also relates to a computer program product comprising a support and stored on this support instructions that can be read by a processor, these instructions being configured to assess the fetal fraction (ff) and sequencing depth (sd) in Non-Invasive Prenatal Testing (NIPT) according to the invention.

The invention also relates to a computer program product comprising a support and stored on this support instructions that can be read by a processor, these instructions being configured to determine the reliability of Non-Invasive Prenatal Testing (NIPT) according to the invention.

The invention also relates to a computer readable medium comprising the computer program product(s) according to the invention.

The features defined above for the methods apply to the computer program products.

### Definitions

The terms used in this specification generally have their ordinary meanings in the art. Certain terms are discussed below, or elsewhere in the present disclosure, to provide. additional guidance in describing the products and methods of the presently disclosed subject matter.

The following definitions apply in the context of the present disclosure:

As used in this specification and the appended claims, the singular forms “a”, “an” and “the” include plural referents unless the content clearly dictates otherwise.

As used in this specification and the appended claims, the term “at least one”, may thus include one, or “more than one”. Accordingly, the terms “a plurality of” or “more than one” may thus include «two» or «two or more».

As used herein, the term “maternal sample” may be from a female at any gestational age suitable for testing, or from a female who is being tested for possible pregnancy, which includes in a non-exhaustive manner any pregnant female at every stage of pregnancy, including a pregnant female subject in the first trimester of pregnancy, in the second trimester of pregnancy, or in the third trimester of pregnancy, for instance a pregnant female between about 1 to about 45 weeks of fetal gestation, which includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 and 45 weeks of fetal gestation (e.g., at 1-4, 4-8, 8-12, 12-16, 16-20, 20-24, 24-28, 28-32, 32-36, 36-40 or 40-44 weeks of fetal gestation). It may also refer to a maternal sample collected during or after (e.g., 0 to 72 hours after) giving birth.

As used herein, the term “maternal biological sample” is referring to any maternal sample, or fraction thereof, which is prone to comprise cell-free DNA. Accordingly, a maternal biological sample may be selected from the group consisting of fluid or tissue samples, including, without limitation, umbilical cord blood, chorionic villi, amniotic fluid, cerebrospinal fluid, spinal fluid, lavage fluid (e.g., bronchoalveolar, gastric, peritoneal, ductal, ear, arthroscopic), biopsy sample (e.g., from pre-implantation embryo), celocentesis sample, fetal nucleated cells or fetal cellular remnants, washings of female reproductive tract, urine, feces, sputum, saliva, nasal mucous, prostate fluid, lavage, semen, lymphatic fluid, bile, tears, sweat, breast milk, breast fluid, embryonic cells, fetal cells (e.g., placental cells), cervical swab, blood, or any fraction thereof including plasma or serum.

As used herein, the term “blood” encompasses whole blood or any fractions of blood, such as serum and plasma as conventionally defined.

As used herein, the term “cell-free DNA”, or cfDNA, refers to DNA that is present in a maternal biological sample (e.g. a maternal plasma sample), which corresponds to a mixture of maternal DNA and fetal DNA. Cell-free fetal DNA (cffDNA) corresponds to the part of cfDNA which corresponds to fetal DNA.

As used herein, the term “fetal fraction (ff)” refers to the percentage of cfDNA in a maternal biological sample (e.g. a maternal plasma sample) of fetoplacental origin, corresponding to the following formula: ff=fetal cfDNA/fetal cfDNA+maternal cfDNA).

As used herein, the term “sequence read” refers to data representing a sequence of nucleotide bases that were measured in a given biological sample (e.g. a maternal plasma sample), for example using a sequencing method (e.g. next-generation sequencing or NGS). In particular, the term “read” may refer to a fragment of contiguous nucleotide sequence (e.g. cell-free DNA as reported above). In a non-exhaustive manner, such reads may be generated from one end of nucleic acids and/or nucleic acid fragments (“single-end reads”), and sometimes are generated from both ends of nucleic acids or nucleic acid fragments (e.g., paired-end reads, double-end reads).

As used herein, the term “sequencing depth (sd)” refers to the average number of times each base pair is sequenced; the sequencing depth increases statistically with the total number of sequencing reads for a given maternal sample. According to some embodiments, the sequencing depth may correspond to a corrected sequencing depth, which corresponds to a normalized sd taking into account the percentage of GC (guanine-cytosine) content by chromosome.

As used herein, the term “sequencing” may refer to all types of sequencing methods, including nucleic acid sequencing carried out with capillary-based, semi-automated implementations of the Sanger biochemistry or any other type of nucleic acid sequencing, in a non-limitative manner, such as high-throughput sequencing. For example, sequencing methods which may be considered herein include Sanger sequencing, sequencing-by-hybridization, nanopore sequencing, pyrosequencing, single-molecule real-time sequencing, Ion semiconductor sequencing, sequencing by synthesis, combinatorial probe anchor synthesis, sequencing by ligation, GenapSys™ sequencing; or else. In a non-exclusive manner, sequencing methods may be applied to amplified nucleic acids (e.g. nucleic acids from a maternal biological sample).

The term “amplified” as used herein refers to subjecting a target nucleic acid in a sample to a process that linearly or exponentially generates amplicon nucleic acids having the same or substantially the same nucleotide sequence as the target nucleic acid, or segment thereof. The term “amplified” as used herein can refer to subjecting a target nucleic acid (e.g., in a sample comprising other nucleic acids) to a process that selectively and linearly or exponentially generates amplicon nucleic acids having the same or substantially the same nucleotide sequence as the target nucleic acid, or segment thereof. The term “amplified” as used herein can refer to subjecting a population of nucleic acids to a process that non-selectively and linearly or exponentially generates amplicon nucleic acids having the same or substantially the same nucleotide sequence as nucleic acids, or segments thereof, that were present in the sample prior to amplification. In certain embodiments the term “amplified” refers to a method that comprises a polymerase chain reaction (PCR).

As used herein, the term “aneuploidy” refers to the state where the wrong number of chromosomes (e.g., the wrong number of full chromosomes or the wrong number of chromosome segments, such as the presence of deletions or duplications of a chromosome segment) is present in a cell. In the case of a somatic human cell it may refer to the case where a cell does not contain 22 pairs of autosomal chromosomes and one pair of sex chromosomes. In the case of a human gamete, it may refer to the case where a cell does not contain one of each of the 23 chromosomes. In the case of a single chromosome type, it may refer to the case where more or less than two homologous but non-identical chromosome copies are present, or where there are two chromosome copies present that originate from the same parent. In some embodiments, the deletion of a chromosome segment is a microdeletion. Aneuploidy can include, for example, monosomy, partial monosomy, trisomy, partial trisomy, tetrasomy, and pentasomy. Examples of aneuploidy that can be detected include Angelman syndrome (15q11.2-q13), cri-du-chat syndrome (5p-), DiGeorge syndrome and Velo-cardiofacial syndrome (22q11.2), Miller-Dicker syndrome (17 p13.3), Prader-Willi syndrome (15q11.2-q13), retinoblastoma (13q14), Smith-Magenis syndrome (17 p11.2), trisomy 13 (Patau syndrome), trisomy 16, trisomy 18 (Edward syndrome), trisomy 21 (Down syndrome), triploidy, Williams syndrome (7q 11.23), and Wolf-Hirschhom syndrome (4p-). Examples of sex chromosome abnormalities that can be detected by methods described herein include, but are not limited to, Kallman syndrome (Xp22.3), steroid sulfate deficiency (STS) (Xp22.3), X-linked ichthyosis (Xp22.3), Klinefelter syndrome (XXY), fragile X syndrome, Turner syndrome, metafemales or trisomy X (XXX syndrome, 47,XXX aneuploidy), and monosomy X.

As used herein, the term “Down syndrome” also known as trisomy 21 refers to a genetic disorder caused by the presence of all or part of a third copy of chromosome 21. It is usually associated with physical growth delays, mild to moderate intellectual disability, and characteristic facial features.

As used herein, the term “Z-score” refers to the standard score used in statistics, and intends to represent the number of standard deviations by which the value of a raw score (i.e., an observed value or data point) is above or below the mean value of what is being observed or measured. Raw scores above the mean preferably have positive standard scores, while those below the mean preferably have negative standard scores.

As used herein, the expression “decision tree” intends to mean a learning model with associated learning algorithms that analyze data, used for classification and regression analysis.

As used herein, the expression “to classify” intends to mean choosing, for a synthetic profile, a group having properties and features representative at least of aneuploid or euploid profiles.

The term “comprise” is to be interpreted as specifying the presence of the stated features, integers, steps or components, but not precluding the presence of one or more other features, integers, steps or components, or group thereof. Also, it may specify strictly the stated features, integers, steps or components, and therefore in such case it may be replaced with “consist of”.

Within the invention, the term ‘significantly” used with respect to change intends to mean that the observed change is noticeable and/or it has a statistic meaning.

Within the invention, the term “substantially” used in conjunction with a feature of the invention intends to define a set of embodiments related to this feature which are largely but not wholly similar to this feature.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Referenced herein may be trade names for components utilized in the present disclosure. The inventors herein do not intend to be limited by materials under any particular trade name. Equivalent materials (e.g., those obtained from a different source under a different name or reference number) to those referenced by trade name may be substituted and utilized in the descriptions herein.

In the description of the various embodiments of the present disclosure, various embodiments or individual features are disclosed. As will be apparent to the ordinarily skilled practitioner, all combinations of such embodiments and features are possible and can result in preferred executions of the present disclosure. While various embodiments and individual features of the present invention have been illustrated and described, various other changes and modifications can be made without departing from the spirit and scope of the invention. As will also be apparent, all combinations of the embodiments and features taught in the present disclosure are possible and can result in preferred executions of the invention.

### EXAMPLES

### Material & Methods

### Patient Cohorts

NIPT was performed on 377 samples from pregnant women (SPW) at Nice university hospital (cohort 1) and 1062 samples at Marseille university hospital (cohort 2) after informed consent. The sequencing data resulting from these series were retrospectively used to validate our bioinformatics suite with consents for research (INDS—MR3310281119; R04-018 Nice and PADS20-53 Marseille). Two non-pregnant women have provided blood as negative control (SNPW). Protocols of DNA extraction, library preparation and sequencing were identical for both cohorts.

### DNA Isolation

Maternal samples were collected in blood collection tubes from Streck (cfDNA BCT) or Roche Diagnostics (Cell-Free DNA Collection Tube) and centrifuged at 1600 g for 10 min to separate the plasma from the blood cells. Plasma was subsequently centrifuged at 16000 g for 10 min. The supernatant was transferred to a new microcentrifuge tube and stored at −80° C. until further processing. cfDNA was extracted from 4 ml of plasma using the QIAamp® Circulating Nucleic Acid kit (Qiagen®, Hilden, Germany) according to the manufacturer's protocol. The DNA was eluted into a final volume of 35 μl of AVE buffer, and concentration was measured using Qubit dsDNA High Sensitivity Kit (Thermo Fisher Scientific) prior to storage −20° C.

### Library Preparation and Sequencing

Shallow whole-genome sequencing of cfDNA was performed using either a Proton or an S5XL sequencer (Thermo Fisher Scientific®), Waltham, MA, USA), starting from 15 ng input of cfDNA. For library building, cfDNA samples were processed either manually or in semi-automated procedure with the Ion Plus fragment library kit and Ion Plus Core Library Module for AB Library Builder TM System respectively (Life Technologies-Thermo Fisher Scientific®, Waltham, MA, USA) using an optimized procedure 6. Library concentrations were measured using Ion Library TaqMan™ Quantitation Kit (Thermo Fisher Scientific). Equimolar concentrations (15 pM) of each library were then automatically prepared and loaded on the chip (IonPI-TM Chip Kit V3 or Ion 540 Chip Kit) using the Ion Chef (Thermo Fisher Scientific®, Waltham, MA, USA). Pre-processing quality control, trimming and mapping to GRCh37 were performed using the Ion Torrent Suite.

### Normalization and Quality Control

Aligned sequences (.BAM) were corrected for GC content using the script gcc.py from Wisecondor, as previously reported in: Straver et al. (“WISECONDOR: detection of fetal aberrations from shallow sequencing maternal plasma based on a within-sample comparison scheme”. Nucleic Acids Res. 2014; 42(5):e31) and WisecondorX, as previously reported in: Raman et al. (“WisecondorX: improved copy number detection for routine shallow whole-genome sequencing”. Nucleic Acids Res. 2019; 47(4):1605-1614).

Each sample was divided in 1 Mb bins. A loess function was then applied with reference to the GRCh37 human genome to obtain normalized GC counts per bin. The normalized read counts are expected around 1. We used the principal component analysis (PCA) to identify samples with unbalanced bin counts, i.e., to be considered as potential library or sequencing quality default, alignment errors or maternal pathology.

iSanefalcon (Optimized Module for ff Estimation)

The stand-alone application is a modified version of the original Sanefalcon (“Single reAds Nucleosome-basEd FetAL fraCtiON») module reported by Straver et al. (“Calculating the fetal fraction for Non-Invasive Prenatal Testing based on genome-wide nucleosome profiles”; Prenatal Diagnosis; 2016, 36, 614-621).

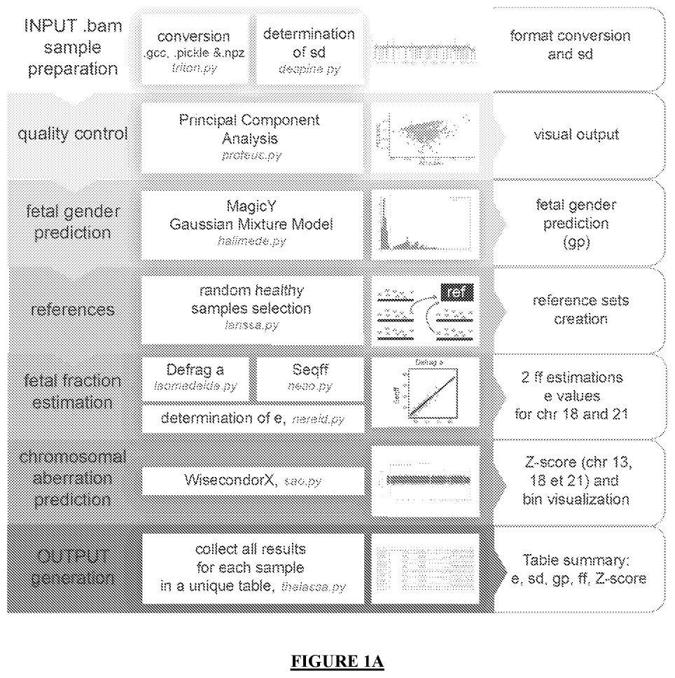

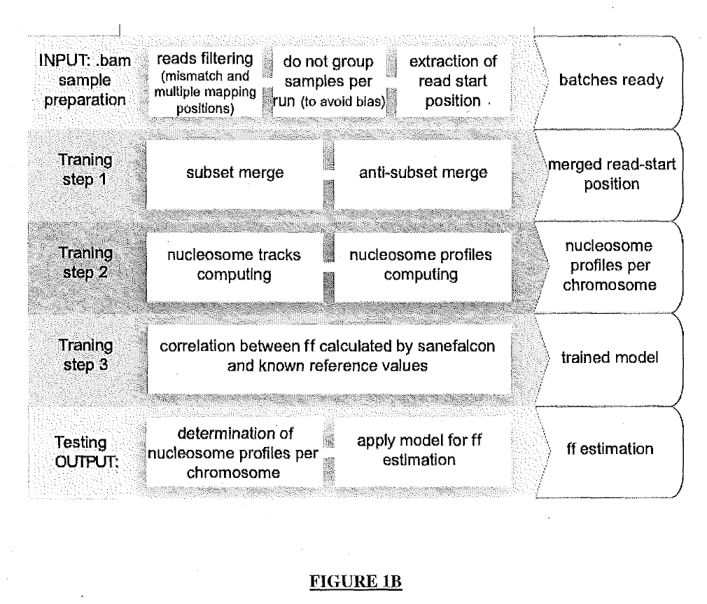

First, we migrated to Python3.6, removing the inner dependencies between Bash scripts and python scripts to make a more stand-alone application, updating the dependencies with the newer versions of supporting software. The stand-alone application can be executed on any platform, and it exploits the core Python libraries for concurrency and parallelization: we heavy parallelized all the steps, from the read-start positions extraction to the nucleosome profile computation. We introduced a dedicated class for managing all the file system operations: in this way there is no manual intervention to be carried out, but everything is managed at runtime. Finally, we reduced at the bare minimum the manual configuration steps that need to be addressed. This way, we made the application more robust and less error prone. A simple configuration file is provided to set up all the locations and the most important parameters for the application. The novel workflow of Sanefalcon is further explained in FIG. 1B.

To demonstrate that iSanefalcon obtains the same results as the original implementation, we calculated ff for both cohorts with both versions, including correlation plots. Around 300 samples for each cohort were used as training set, the rest as test, having care to keep together samples from the same run. To provide ff for each sample, the procedure was applied 5 times for cohort 1 and 2 times for cohort 2. Good correlation values are reported for both confirming that the two versions of Sanefalcon do not show significant difference on ff calculation. However different trends are observed for cohort 1 only. To inspect this result, we used a mixed effect model with the equation:

$$y = ax + b + r$$

combining the linear model expected with a random effect r, that cause the offsets on Y axis for each group. Both the likelihood and Akaike Information Criterion (AIC) of linear and mixed model confirmed that the mixed model better suits the modeling for cohort 1 (linear model, Log-likelihood:-537.31 AIC: 819.906; mixed model, Log-likelihood: 735.863 AIC:-1463.727). The of mixed effect is relevant only for cohort 1 ruling out that it is due to the new implementation of Sanefalcon. PCA analysis on both cohort suggests the mixed effect for cohort 1 does not originate from the samples. Sanefalcon strategy is the only analysis done at read start position resolution, while the others are the bin level. This suggests that the read start position resolution is not appropriate for small-size cohorts, probably due to a lack of variability.

### NiPTUNE

The complete suite for NIPT, upon which ff and sd are then assessed, includes a selection of previously reported blocks/modules, some of which were modified as described hereafter and in FIG. 1A. The following paragraphs explain each block composition and purposes.

### Configurations and Input Files Preprocessing

This block, composed of two modules, prepares sample files for use by downstream tools of the pipeline and calculates sd.

The module triton.py converts the original file format (.bam) to other formats (.gcc, .pickle, .npz) which serve as input for downstream processing in NiPTUNE. The “.pickle” output contains aligned reads split in 1 Mb bins (tunable parameter), the “.gcc” output is the result of GC correction at the bin level obtained by applying a lowess model with respect to the reference genome. Finally, the “.npz” format is a specific file used as input for WisecondorX. The module despina.py calculates the sd of the sample(s) provided.

### Quality Control

The module proteus.py performs a principal component analysis on all the samples of the cohort and produce a visual output to check whether there are outlier samples or subpopulations into the cohort. We used the function prcomp of R software.

### Fetal Gender Prediction

To predict the gender of the fetus the module halimede.py implements a novel method (referred herein as “MagicY”) that first quantifies the proportion of reads of seven Y-specific regions with respect to the number of reads on the Y chromosome:

$$MagicY = \frac{\sum \# reads specific region ChrY}{\# reads on ChrY} .$$

The seven Y-specific regions which are considered herein are listed in Table 1:

TABLE 1
list of Y-specific regions considered for fetal gender prediction.
Y chromosome specific regions

| Name | Chromosome | Start | End | Gene name |
|---|---|---|---|---|
| R1 | chrY | 20708557 | 20750849 | HSFY1 |
| R2 | chrY | 25119966 | 25151612 | BPY2 |
| R3 | chrY | 26753707 | 26785354 | BPY2B |
| R4 | chrY | 27177048 | 27208695 | BPY2C |
| R5 | chrY | 19880860 | 19889280 | XKRY |
| R6 | chrY | 24636544 | 24660784 | PRY |
| R7 | chrY | 24217903 | 24242154 | PRY2 |

Then, it is combined with a gaussian mixture model with two gaussians used to fit the distribution of the ratio of counts and to identify the threshold to separate male and female populations. We implemented the same gaussian mixture model using the function GaussianMixture from Python package sklearn (version 0.23.1) to predict gender on the fetal fraction (ff) calculated by the algorithmn reported in Bayindir et al. (“Non-Invasive Prenatal Testing using a novel analysis pipeline to screen for all autosomal fetal aneuploidies improves pregnancy management”. Eur J Hum Genet. 2015; 23(10):1286-1293.

WisecondorX quantifies the proportion of reads on the Y chromosome with respect to the total number of reads per sample. To identify the threshold to discriminate fetal gender, we projected MagicY threshold on y fraction counts. Finally, Defrag uses a KNN binary classification on a training set to classify samples as males or females

### Reference Sets Creation

The module larissa.py randomly selects n samples to be used as reference for Defrag and WisecondorX. In the present work, we set n=100 samples for each cohort. We strongly recommend the use of cohort specific samples to improve precision and reliability of the different tests. The module prepares reference samples to be used by downstream programs (i.e. Defrag a, WisecondorX).

### Fetal Fraction Prediction

Performances of four ff estimation tools were assessed on our two cohorts:
Defrag a and Defrag b were previously described in Beek et al. (“Comparing methods for fetal fraction determination and quality control of NIPT samples”. Prenatal Diagnosis. 2017; 37(8):769-773).
Seqff was previously described in Kim et al. (“Determination of fetal DNA fraction from the plasma of pregnant women using sequence read counts: Determination of fetal DNA fraction from the plasma of pregnant women using sequence read counts”. Prenatal Diagnosis. 2015; 35(8):810-815).
Sanefalcon and its optimized module iSanefalcon, as previously stated.

Default parameters were used for Defrag a, Defrag b and Seqff,

Two methods are proposed in NiPTUNE to calculate ff, namely: Defrag a based on chromosome Y read counts, implemented in the module laomedeida.py and Seqff based on pre-trained bin counts in the module neso.py. The main code of Defrag and Seqff were maintained in NiPTUNE. We added a script to parse Seqff input to make it more efficient and able to calculate ff on multiple samples as in the original implementation and on single sample. Sanefalcon is not implemented in the main workflow because our benchmark demonstrated that it performs less efficiently on our samples. We provide an improved version of this tool to allow users to run it independently of NiPTUNE.

Finally, a module to calculate the E value is present, nereid.py. The E value represents the estimated contribution of aneuploidy to ff and is chromosome-specific. The module calculates two values, one for chromosome 18 and one for chromosome 21, (validated for our cohort). For a detailed explanation of the E value, please refer to paragraph “Modeling chromosome-specific contribution to ff”.

### Copy Number Alteration Prediction

We implemented WisecondorX in the module sao.py. For each sample submitted to this module, a global Z-score is calculated on the binned sample with respect to the reference samples. A graphical output is also provided to visually inspect the chromosomes with abnormal counts.

### Prepare Output

This last module of the pipeline, thalassa.py, collects results from upstream modules in a table. Each sample (line) is described by the following columns: quality control (visual output), E value for chromosome 18 (chr18), E value for chr21, sd, gender prediction (magicY), ff (2 columns one for each method) and the Z-score for chromosome 13, 18 and 21.

### GenomeMixer (Novel Module to Create Synthetic Sequencing)

In order to study how ff and sd impact the prediction of chromosomal abnormalities, we established a strategy to increase the number of aneuploid samples at our disposal. Specifically, we needed to modulate either the ff or sd of the sequencing input to identify minimal thresholds for these two parameters. We reasoned to set up a bioinformatic tool to create the missing samples based on two strategies.

On one hand, to create synthetic sequencing with lower ff (GenomeMixer_ff), reads from the original alignment file need to be replaced by reads from a control file, the Samples from Non-Pregnant Women (SNPW). Specifically, in order to reduce the ff while keeping a stable sd, the reads to be replaced need to originate from the fetal genome.

On the other hand, to decrease the sd (GenomeMixer_sd), reads to be removed should belong to both maternal and fetal populations while keeping this ratio unchanged. It is, however, impossible to clearly distinguish fragments of maternal or fetal origin.

We thus reasoned to associate to each read a weight that represents its propensity to be coming from the fetal or maternal DNA.

It is accepted in the literature, that fragments of fetal origin are shorter than maternal ones, as previously reported in Lo (Non-invasive prenatal testing using massively parallel sequencing of maternal plasma DNA: from molecular karyotyping to fetal whole-genome sequencing. 2013. Reprod. Biomed. Online, 27, 593-598) and Chiu et al. (Non-Invasive Prenatal diagnosis of fetal chromosomal aneuploidy by massively parallel genomic sequencing of DNA in maternal plasma. 2008. Proc. Natl. Acad. Sci., 105, 20458-20463).

We confirmed this observation on euploid samples from our cohorts and decided to take advantage of this property to calculate the weights to be associated to each read. Briefly, we merged all aneuploid T18 samples, proceeded similarly for T21 and SNPW and calculated the reads length distributions. These three distributions represent the “reference distributions” for each category.

Then, we calculated the difference between the reference distributions associated to each trisomy pool (in this case T18 and T21) and the SNPW. These curves represent the quantification of the difference in the frequency of each read length between the aneuploid sample pools and SNPW.

To maximize the difference between the two distributions, we applied a step function. The amplitude of the step corresponds to the values between the minimal local maximum of the difference curve. All reads having the same length, will be labeled with equal weights. The weights allow to prioritize the selection of reads belonging to the fetal population prior to maternal ones to be replaced for GenomeMixer_ff, and to maintain fetal/maternal read balance while removing reads for GenomeMixer_sd.

In order to apply our strategies to build synthetic sequencing, we performed a weighted probability sampling on the sequenced genomes presenting a chromosomal aberration. We prioritized reads from the putative fetal fragment population as candidate reads to be replaced or removed, depending on the parameter (i.e. ff or sd) to be modeled. The SNPW are used only for GenomeMixer_ff. Each sampling is done at chromosomal level. The amount of reads to be replaced or removed is a user-defined percentage, however the two strategies are slightly different.

For GenomeMixer_ff, the reads to be replaced from the Samples from Pregnant Women (SPW) with fetal aberration are selected from the Samples from Non-Pregnant Women (SNPW) using the the weights calculated as explained earlier. On the aneuploid chromosome, half of the reads sampled from the fetal population are replaced and half of them are suppressed (see paragraph “Modeling chromosome-specific contribution to ff” for detailed explication). At the end of the process, the result is a synthetic sequencing with the same number of reads than the original one, with an error of less than 0.001%, but coming from different sources.

For GenomeMixer_sd, in order to keep the ff stable while lowering the sd, the reads to be removed are selected respecting the proportion of ff. For instance, if we want to remove 100 reads and the ff is 10%, thus we remove 10 reads from the reads labeled as most likely belonging to the fetal population and 90 reads labeled with the opposite weights.

### Modeling Chromosome-Specific Contribution to ff

Budis et al. (“Combining count-and length-based Z-scores leads to improved predictions in non-invasive prenatal testing”. 2019. Bioinformatics, 35, 1284-1291) defined lambda-score profiles using progressive elimination of fragment based on several length limits. They showed that the lambda scores of aneuploid samples deviates from euploids, leading to the idea that there is an extra contribution of fetal reads of aneuploid chromosomes compared to euploid samples. We used this property, to define the E value to improve the prediction accuracy.

In the previous paragraph we observed that SPWs are enriched in reads of a specific length range compared to SNPW (fetal_range). Thus we reasoned that we can approximate the contribution of fetal reads to the read count on a chromosome as the number of reads with length in the fetal_range times the ff: ff*n_{(fetal_range)}^{chrT}.

On the other hand, based on the assumption that fetal reads are randomly distributed on the genome, we reasoned that the fetal reads originating from a chromosome can be estimated as the product of the number of reads in the fetal_range times the ff, and the proportion of reads on the chromosome of interest.

This proportion is defined as the number of reads on the chromosome of interest (T) divided by the total number of reads:

$${p}_{chrT} = \frac{n reads chrT}{n reads genome} .$$

We can model fetal reads as uniform random draw, thus it is represented by a binomial distribution with variance v_{chrT}=n_{(fetal_range)}^{genome}*ff*p_{chrT}(1−ff*p_{chrT})).

The E value is defined as:

$${E}_{ChrT} = \frac{ff * {n}_{( fetal \_ range )}^{chrT} - {n}_{( fetal \_ range )}^{genome} * ff * {p}_{chrT}}{\sqrt{{n}_{( fetal \_ range )}^{genome} * ff * {p}_{chrT} ( 1 - ff * {p}_{chrT} )}} \\ Where \\ {p}_{chrT} = \frac{n reads chrT}{n reads genome} .$$

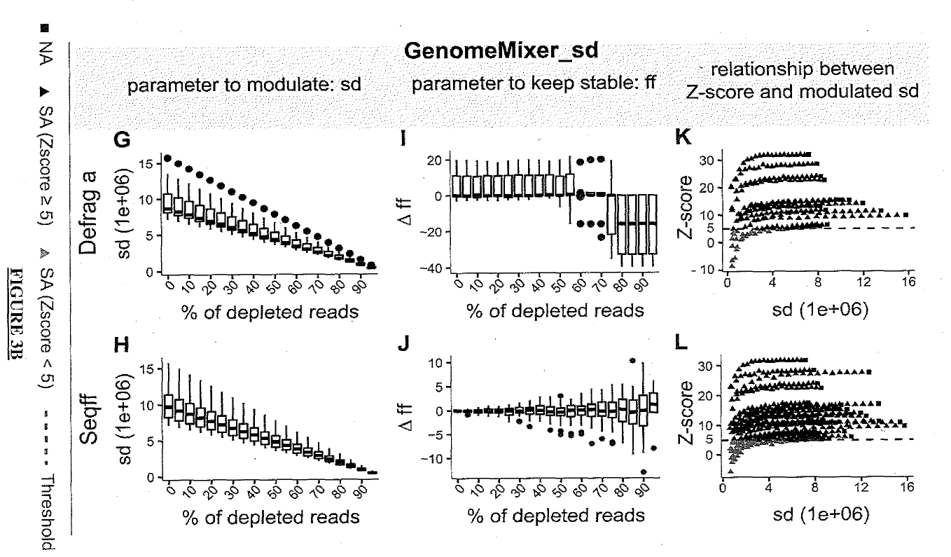

The more the E value deviates from 0 the likelier the chromosome T is to present an anomaly, as shown in FIG. 4, where aneuploid samples from the two cohorts are shown for chromosome 18 and chromosome 21.

### Synthetic Sample Generation

In order to study how the ff impacts the prediction of chromosomal aberrations, we used GenomeMixer_ff to create samples with increasingly lower ff but with constant number of reads. GenomeMixer_ff takes as input SPW with confirmed fetal trisomy and SNPW. For each SPW with fetal trisomy, we generated 19 new samples by replacing increments of 5% of the initial reads counts of the SPW with the equivalent amount from the SNPW (see materials and methods for detailed explanation about the criteria used to select reads to be replaced/to replace). The small number (2) of twin aneuploid samples did not allow us to validate our model on twin pregnancies. We used SPW samples with either T21 or T18 identified by a Z-score ≥5 to feed GenomeMixer. Thus, from 23 native aneuploid (NA) samples with fetal T21 and 7 NA samples with T18, we generated respectively 437 and 133 synthetic aneuploidies (SA). Seqff estimated ff for all SA obtaining ranges from 0.88 to 35.5. Defrag a estimated ff for 197 out of 345 SA originating from NA male fetuses. The ff minimal value range from 3.04 to 37.91.

To evaluate the impact of sd on chromosomal abnormalities prediction, we used GenomeMixer_sd. It takes as input only SPW with fetal chromosomal aberrations. In order to generate new samples with increasingly lower sd, we removed increments of 5% of initial reads counts while keeping the ratio between fetal and maternal reads stable. We iterated this process 19 times for each NA obtaining 437 and 133 SA, with a sd range from 360261 to 15002811. Both Seqff and Defrag a could not estimate ff for the totality of SA generated with GenomeMixer_sd (28/670 for Seqff and 154/345 for Defrag a).

Altogether our results show that Seqff estimates ff even for very low values, while Defrag a could not for ff lower than 3.

### TRUST

We implemented a web application called TRUST: Trisomy Reliability Unique Score Test, to test the reliability of NIPT test based on the values of the parameters: ff, sd and e. Using the decision trees, the application calculates the reliability score (Rscore) and classifies the NIPT results as:
“highly reliable”: Rscore is between 0.8 and 1. Sd, ff and e provided for the samples fulfill the required values to achieve a reliable prediction.
“reliable”: Rscore is between 0.2 and 0.8. One or more parameters are below the threshold, thus a potential abnormality might be missed by the Z-score calculation. In this case, redo the sampling can be considered if a higher level of accuracy want to be achieved.
“not reliable”: Rscore is between 0 and 0.2. Parameters do fulfil the required standards, thus abnormality assessment by Z-score calculate on is not reliable. New sampling is strongly advised.

### Statistical Analysis

All statistics were performed using the software R. Violin plots were done using the library ggplot2 from R. Correlations were calculated using corr function from R.

### Results

### Sequencing Quality Control

NIPT was performed on 2 cohorts of pregnant women: Nice (cohort 1) and Marseille (cohort 2). Cohort 1 consists of 377 samples, including 11 fetal aneuploidies. Cohort 2 is composed by 1062 samples, including 20 fetal aneuploidies (Table 1). To verify that samples do not have aberrant read count distributions, we applied the principal component analysis (PCA) on binned count of normalized reads for samples belonging to each cohort. We observed that the distribution of points (samples) is coherent for the two cohorts: most of the points are clustered in the centroid of the plots. If cohorts came from two different hospitals, the extraction and sequencing method was identical. We thus joined the two cohorts for cross comparison. Sequencing results from 2 SNPW, added as controls, are distributed homogeneously, alike fetal aneuploidies. To test the reliability of the method, we also added 3 test samples from sequencing results to those from cohort 1. Two of them resulted from failed alignments to the reference genome and the third one corresponded to a maternal aneuploidy. The points corresponding to the 3 test samples were scattered from the main group. These analyses support the validity and importance of using PCA as a quality control to test the distribution of read counts after mapping before further analysis. Furthermore, they highlight the contribution of PCA to identify disease-associated genomic maternal abnormalities that could lead to false NIPT interpretation.

### Identification of a Reliable Strategy for Fetal Gender Prediction

Fetal gender prediction is an important step of NIPT pipeline because the gender of the fetus is used by several tools to determine the set of samples to be used as reference. Thus we needed to establish a confident method to predict fetal gender from sequencing data. We tested different tools described in the literature and we compared them with a novel method that we developed called “MagicY”, based on chromosome Y-specific regions, followed by a gaussian mixture model approach to estimate fetal gender (Supplementary Information). We showed that MagicY outperforms the tested methods.

### Benchmark of Tools for ff Estimation

The ff estimation is a fundamental parameter for reliability of chromosomal anomaly calculation. If aneuploidy is ff-independent per se, its prediction can be affected by a low ff⁶. Despite the importance of ff, a gold standard method for its calculation is not yet established. We have compared the performances of four tools most commonly used in the literature: Defrag a, Defrag b, Seqff and Sanefalcon.

Although Defrag b shows stable results for the two cohorts, our benchmark demonstrated that this tool underestimates low ff and overestimates high ff. iSanefalcon showed both strong cohort-dependent behaviors and very poor correlation with any other tool for ff calculation. Thus, we selected Defrag a and Seqff as confident tools for ff calculation.

### Computational Prediction of Chromosomal Aberrations

To test for fetal aneuploidies, we used WisecondorX, an upgrade of the original version Wisecondor. A Z-score greater than 5 is indicative of a potential chromosomal abnormality. Cohort 1 is composed by 11 aneuploid samples, including 5 trisomies of chromosome 18 (T18) and 6 trisomies of chromosome 21 (T21). Cohort 2 contains 20 ancuploidies, among which two T18, sixteen T21, one sample with both T18 and T21 and one T13. Among aneuploidy samples, 2 corresponded to dichorionic diamniotic twin pregnancies with one fetus out of two carrying either a T18 or a T21.

The Z-scores calculation with WisecondorX identified all trisomies in both cohorts, including the ones for twin pregnancies and the double trisomy. Furthermore, WisecondorX did not lead to false negative results for chromosomes 13, 18 and 21. These results confirm the specificity and the sensibility of WisecondorX approach.

### NiPTUNE: a Computational Pipeline to Perform NIPT in an AccUrate, INtegrative and FlexiblE Framework

NEPTUNE can be used on any data regardless of sequencing technology. We demonstrated that the Principal Component Analysis (PCA) is a valid quality control, thus we implemented it as a first step before any other calculation. We provide a module that estimates the gender of the fetus with Magic Y. NiPTUNE offers estimations of ff with two tools, namely Defrag a and Seqff and chromosomal aberrations are assessed by WisecondorX. Finally, we generated a module that automatically collects all the results of analyzed samples in a table-like format that can be easily processed. Of note, NiPTUNE can run either a single sample or batches of samples.

### GenomeMixer: a Novel Bioinformatic Tool to Create Synthetic Sequencing of Pregnant Women

We have shown that bioinformatics tools to estimate ff produce very different results. Thus, ff values in different clinical laboratories are not comparable and a gold standard threshold of ff to validate NIPT results cannot be determined. Moreover, a sufficient ff is needed to avoid false negative results. Higher sd could compensate for low ff but a clear description of this relationship is missing. We aimed at providing labs with a reliable way to establish confidence intervals for NIPT, specifically the minimal ff and sd necessary to predict chromosomal abnormalities with confidence. However, the determination of these minimal values requires a very large range of both ff and sd in aneuploid samples, very difficult to obtain. in clinical practice.

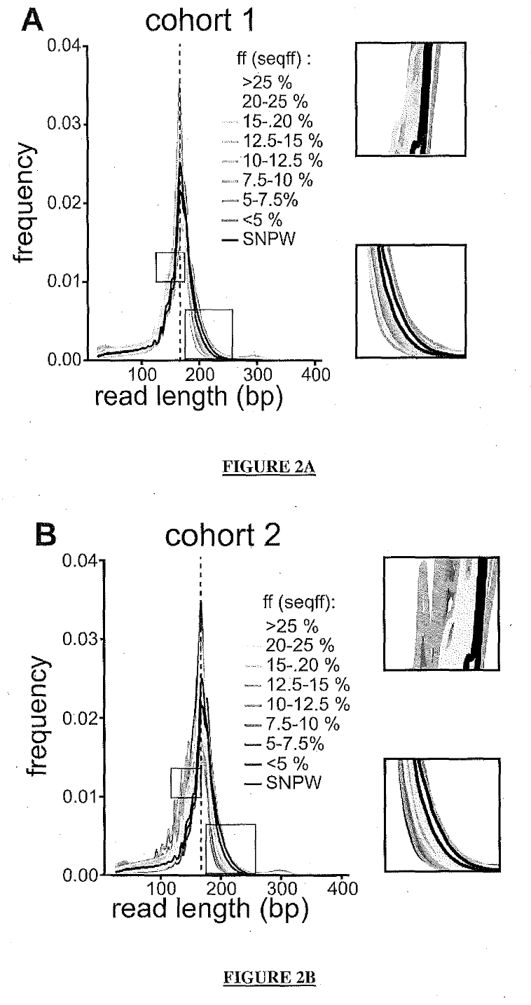

Thus we developed GenomeMixer, a semi-supervised data augmentation approach that generates new synthetic samples while controlling the ff (i.e. GenomeMixer_ff) or the sd (i.e. GenomeMixer_sd). Briefly, GenomeMixer creates synthetic alignment files mixing sequencing reads, from “native” samples from pregnant women (SPW) with fetal confirmed ancuploidies and from non-pregnant women (SNPW) in order to modulate either the ff, keeping the number of reads stable, or the sd keeping the ff stable. The cfDNA in pregnant woman plasma is a mixture of fragments either belonging to the mother or the fetus. Thus, there is no way to easily distinguish their origin. One of the properties that can be used to label the reads is their length. It is established that the population of fetal cfDNA is enriched of smaller fragments compared to the maternal ones with a main fetal peak around 143 bp and the maternal one around 166 bp. We observed this pattern when we calculated the read length distributions for our cohorts (FIGS. 2A-2B). The “maternal” peak, found for our cohorts at 167 bp, is preceded by a shouldering composed of shorter fragments of potential fetal origin. FIG. 2A shows that, depending on the ff, read length distributions are quite different: at low ff values correspond a greater number of long reads. The highest peak is indeed observed for lower ff values. As the ff increases, this peak decreases concomitantly to an increase of the number of shorter fragments. We reasoned that we could associate a weight to a fragment length, thus representing the likelihood for the fragments to belong to one or the other population (maternal or fetal). The workflow of GenomeMixer is fully described in FIG. 2C. This program allowed us to generate synthetic samples necessary to establish quality thresholds for NIPT reliability.

### The Impact of ff and sd on Fetal Chromosomal Aberration Prediction

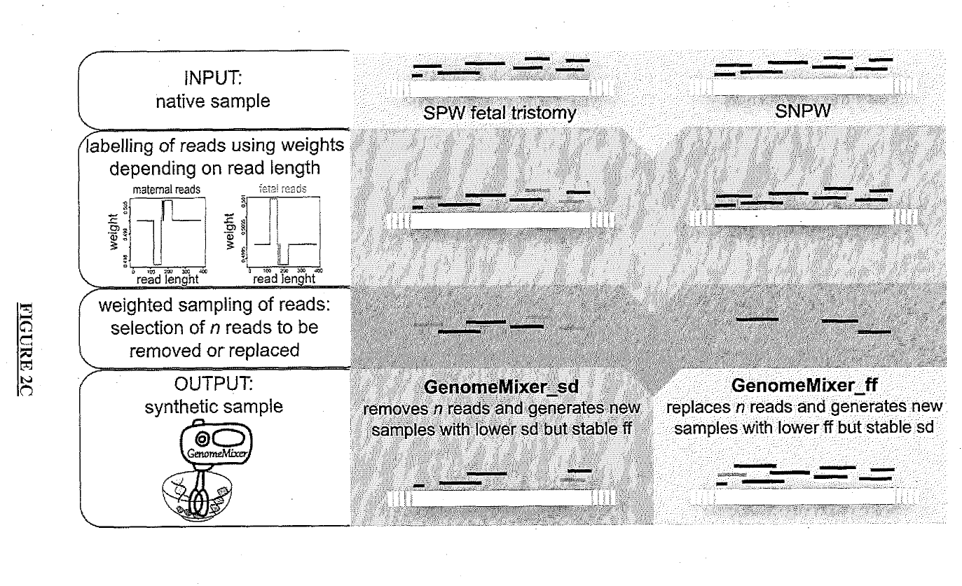

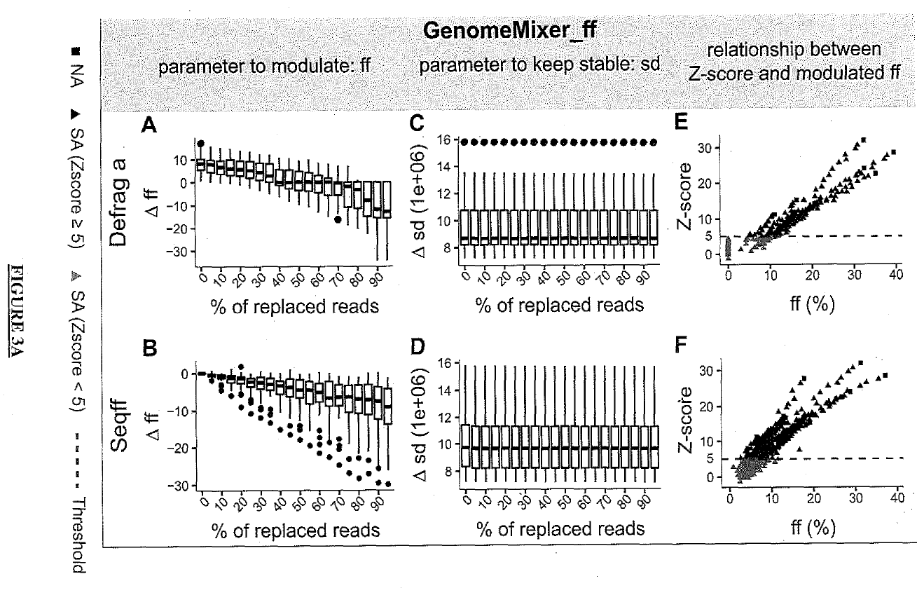

FIGS. 3A and 3B report results for all samples generated by GenomeMixer, including both T18 and T21, with ff values calculated with Defrag a or Seqff. Overall, ff of samples generated with GenomeMixer_ff decreases consistently with the percentage of replaced reads (FIG. 3A), while sd does not change (FIG. 3B). With a same percentage of removed reads, we expect a proportional decrease of ff for all samples. If this relationship is observed with the analysis by Defrag a, surprisingly, this proportionality is not found with Seqff for high percentages of replaced reads. This variability suggests that ff calculation with Seqff becomes less reliable for samples with low ff.

Then, we calculated the Z-scores for SA and plotted the Z-scores versus the ff for all samples (native and synthetic). A linear relationship between the Z-score and ff is found, either calculated with Defrag a (correlation: spearman 0.96, pearson 0.94) or Seqff (correlation: spearman 0.88, pearson 0.92). This analysis demonstrates that the estimation of fetal aneuploidies strongly depends on the ff found in sequenced samples. Furthermore, we observed SA with low ff not called as aneuploid (Z-score less than 5), highlighting the importance to find a threshold of minimal ff needed to achieve a reliable prediction of chromosomal aberrations.

Using the same strategy, we validated that samples generated with GenomeMixer_sd had increasingly lower sd (FIG. 3G-H). As expected, no significant variation of ff calculated with Defrag a, was observed. By contrast, the reliability of ff calculation with Seqff decreases proportionally with the number of depleted reads, suggesting that sd impacts the validity of ff calculation by Seqff. Finally, we plotted the relationship between the Z-scores for SA and NA, and the sd. We observed two trends: a flat behavior of Z-scores while depleting reads until a limiting value is reached after which the Z-scores dramatically drop. This result suggests that Z-score calculation is quite robust regarding sd. However, the Z-score is not able to identify with confidence aberrant samples for extremely low sd.

For the first time, we provided an analysis of the relationship between Z-scores and either ff or sd. Seqff appears less reliable than Defrag a for calculation of low ff values, increasing the difficulty to determine a threshold for a minimal ff value needed to guaranty reliable NIPT. On the contrary, Z-scores seem less affected by sd. However, when the Z-score for NA is around the threshold of 5, the decrease of sd shortly leads to a drop of the Z-score and a false negative result. Altogether, our data highlight the interdependence between ff, sd and Z-scores.

### Assessment of Confidence Intervals for Reliable NIPT for Clinical Practice

We set up a decision tree based approach (see materials and methods) to find the relationship between sd, ff and Z-score. We decided to include the E value, corresponding to the chromosomal specific contribution to ff, because it can help to classify samples.

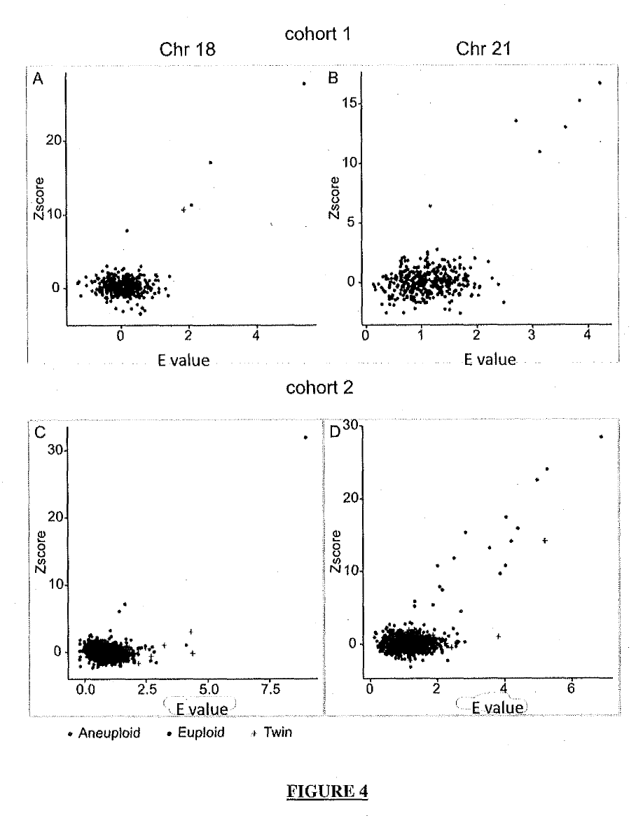

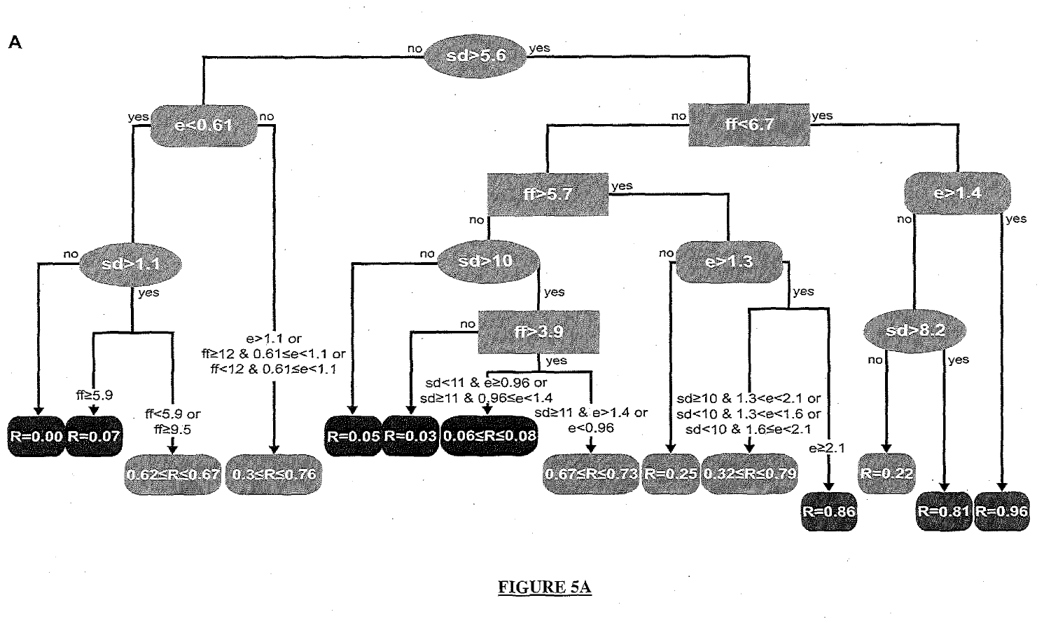

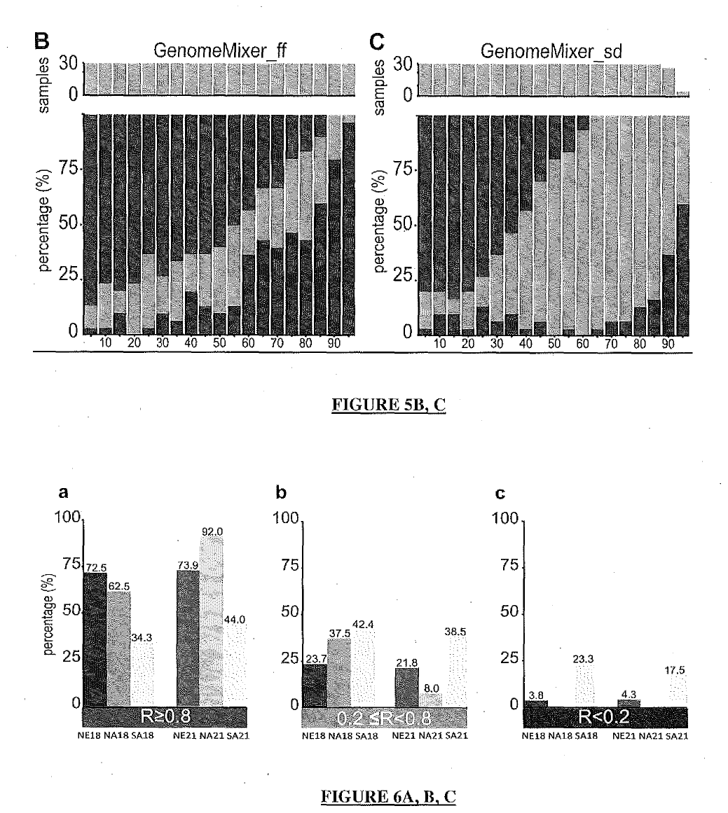

We used NA and SA generated with GenomeMixer, classified by Z-score, to feed a decision tree. Groups of samples were isolated based on combinations of ff, sd and e. We run the decision tree approach using the ff estimated either by Seqff, or by Defrag a in order to identify a minimal threshold for ff, sd and e specific to each tool. FIG. 4A reports the results of the decision tree approach using Seqff. We observed several levels of classification: the first divides samples based on their sd, with a discriminant threshold of 5.6 millions of reads. The second level groups samples with higher sd than the previous discriminant threshold, based on their ff with a discriminant value of 6.7%. Samples with lower sd than the threshold in level 1 are grouped based on their E value, with a discriminant threshold of 0.61. The following levels depend on different combinations of sd, ff and e. Finally, 14 combinations of parameters were found to stratify samples (FIG. 5A, B, C). The same approach was used with Defrag a.

To facilitate the stratification of samples, we defined a reliability score, the Rscore, associated to samples belonging to each of these groups, that represents the probability that the prediction of the aneuploidy based on the Z-score calculation is reliable regarding the value of ff, sd and e. Rscore values go from 0 to 1. For ease of usage, we defined three categories: “highly reliable” when Rscore is between 0.8 and 1; “reliable” when Rscore is between 0.2 and 0.8 and “not reliable” when Rscore is lower than 0.2.

### TRUST: a Web Application That Attributes a Reliability Score to NIPT Results

To test the identified confidence intervals on our cohorts, including native euploid (NE), NA and SA generated with GenomeMixer, we developed TRUST, Trisomy Reliability Unique Score Test, a web application that attributes a chromosome-specific Rscore to NIPT results.

First, we focused on SA. We demonstrated that the lower the ff or sd, greater the count of Rscores belonging to a “not reliable” category. This result reinforces the importance of these 2 parameters in NIPT reliability.

For SA generated by GenomeMixer_ff, 60% of replaced reads result in “not reliable” NIPT outcome in 3% of cases. When more than 85% of reads are replaced, it increases to more than 50% (FIG. 5B). By contrast, for SA generated with GenomeMixer_sd, when 85% of reads are depleted, a “not reliable” score is obtained in less than 20% of cases (FIG. 5C). Overall, this analysis indicates that the reliability of the test is more affected by low ff than by low sd, independently of the tool used to estimate ff.

Most of the native samples, both euploid and aneuploid (72.5% NE18 and 73.9% NE21; 62.5% NA18 and 92% NA21) fall in the confidence interval with the highest Rscore (R>=0.8) for Seqff tree (FIG. 6A). A smaller percentage of NE samples is classified in the intermediate level (“reliable”): 23.7% NE18 and 21.8% NE21, for Seqff and 13.3% NE18 and 13.3% NE21, for Defrag a. Only 3 NA18 and 2 NA21 are classified as “reliable” in Seqff decision tree. The 3 NA18 samples have a low ff (1_240: 4.25%, 2_477: 4.96%, 1_40: 6.42%). Both samples NA21 are classified as “reliable” in the Defrag a tree as well.

This result is due to the sd of the two samples: 8162972 and 8819954 for samples 2_1012 and 1_128, respectively. The sample 2_1012 carries also a T18 and is classified as “reliable” with Defrag a decision tree while it has a “highly reliable” outcome for Seqff. The difference in the Rscore outcome is due to the E value that plays a less important role in Defrag a tree compared to the Seqff one. Importantly, none of the NA samples in both trees, very few NE samples (less than 4%) for Seqff and none for Defrag a, are classified as “not reliable” (FIG. 6C).

This result highlights the contribution of TRUST in spotting problematic samples undetected by classical methods. It helps decreasing false negative results rate and improves the reliability of NIPT by identifying the deficient parameter and its specific correction (i.e. additional sample sequencing or novel blood test). We showed that the E value can help stratifying samples especially for low ff and/or low sd. It had been suggested that higher sd could compensate for low ff. Our data showed that this parameter can be used to improve test reliability in case of low ff values.

### Identification of a Reliable Strategy for Fetal Gender Prediction

Fetal gender prediction is an important step of NIPT pipeline because the gender of the fetus is used by several tools to determine the set of samples to be used as reference. Thus we needed to establish a confident method to predict fetal gender from sequencing data. We tested different tools described in the literature on cohort 1, including 377 samples. Gender could not be confirmed for 28 samples out of 377 because we could not obtain the pregnancy outcome for these fetuses (with no chromosomal abnormality according to NIPT). The analysis was not performed on cohort 2 because gender outcome was not available.

We defined a new calculation that we called “MagicY”. We selected 7 chromosome Y-specific regions and estimated the proportion of reads belonging to these regions (Table 1). A bimodal distribution is observed. Thus, to separate male and female samples, we applied a gaussian mixture model on the distribution of these counts for the entire cohort. This method gave an agreement of 97.1% with gender outcomes, respectively.

To validate our approach, we compared with other three available methods. First, we used the calculation based on autosomes and chromosome X, proposed by Bayindir et al., as part of their aneuploidy detection pipeline. Beek and colleagues suggested that Bayindir was not the most reliable tool for ff calculation. Our data agree with theirs, we observed that ff calculation stratifies samples by gender (p.value<2.2 10⁻¹⁶ Wilcoxon test). We thus resolved to use this property for gender prediction. We then applied the gaussian mixture model to Bayindir results and obtained an agreement of 94.3% with gender outcomes. The suite of tools from WisecondorX predicts fetal gender by calculating the proportion of reads on chromosome Y with respect to the total number of reads for each sample followed by a gaussian mixture model. However the model could not find a threshold to separate male and female populations. Since gender prediction is a mandatory step for WisecondorX, we needed to find another strategy to assess this threshold.

Thus we projected the threshold estimated by the mixture model from MagicY counts of y fraction counts obtained by WisecondorX. This procedure gave an agreement with pregnancy outcomes of 87.7%. This lower agreement could be due to pseudoautosomal regions of X and Y chromosomes leading to a miscount of chromosome Y-specific reads. The Defrag ff estimation tool includes a gender prediction step. Analysis of cohort 1 by Defrag only agreed with 90.3% of gender outcomes (Supplementary Table 2).

In conclusion, these analyses allowed us to develop a novel read count, based on chromosome Y-specific regions, followed by a gaussian mixture model approach to estimate fetal gender with high accuracy called MagicY.

### Assessment of Confidence Intervals for Reliable NIPT for Clinical Practice

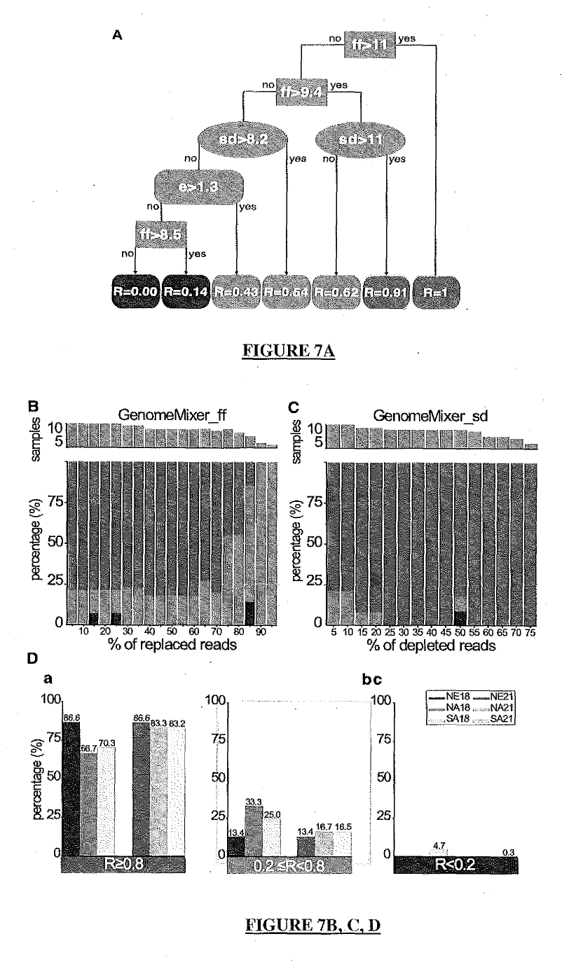

The same approach was used with Defrag a (see FIG. 7). Five levels were identified. The first divides samples based on their ff, with a threshold value of 11%. For the second level, the ff discriminant value is of 9.4%. The subsequent level is defined by the sd. For ff higher than 9.4%, the sd of 11 milions of reads separates samples. For ff smaller that 9.4%, samples are further grouped based on their sd, with a threshold of 8.2 millions of reads. Samples in this last level are further grouped based by their E value (threshold of 1.3) and then by their ff (threshold of 8.5%). In total, seven combinations of parameters where determined to stratify samples. This decision tree has fewer combinations than the previous one. This could be explained by the lower complexity of the samples analyzed. Defrag a fails to estimate ff for both low sd and ff, and provides results for males only. Of note, the E value appears in the Defrag a tree only at the forth level, while in the Seqff one it plays an important role already at the second level, suggesting that samples can be classified differently by the two trees.

To facilitate the stratification of samples, we defined a reliability score, the Rscore, associated to samples belonging to each of these groups, that represents the probability that the prediction of the aneuploidy based on the Z-score calculation is reliable regarding the value of ff, sd and e. Rscore values go from 0 to 1. For ease of usage, we defined three categories: “highly reliable” when Rscore is between 0.8 and 1; “reliable” when Rscore.

1. A method for assessing the fetal fraction (ff) and sequencing depth (sd) in Non-Invasive Prenatal Testing (NIPT), comprising the steps of:
a) providing a set of sequences reads from a maternal biological sample, wherein for each sequence read the fragment length is known,
b) assigning a weight factor to one or more fragment length(s) of the set of sequences reads,
c) computing a synthetic profile, whereby a selection of at least one sequence read from the maternal biological sample is removed, or replaced with a selection of sequence read(s) from non-pregnant sample(s),
d) computing a value (E) corresponding to the synthetic profile, based on at least one of (i) the total number of reads with fragment length in a reference fetal range and (ii) the number of reads with fragment length in a chromosome of interest (T) in a reference fetal range, and
e) estimating the fetal fraction and sequencing depth of the synthetic profile based at least on said value (E).

2. The method according to claim 1, further comprising a step of isolating cell-free DNA (cfDNA) from the maternal biological sample.

3. The method according to claim 1, further comprising a step of sequencing isolated cell-free DNA (cfDNA) from the maternal biological sample, thereby obtaining a set of sequence reads.

4. The method according to claim 1, comprising a step of removing at least one read from the sequences reads, thereby generating a synthetic profile with a modulated sequencing depth (sd) compared to the synthetic profile of step c).

5. The method according to claim 1, comprising a step of replacing at least one read from the sequences reads, thereby generating a synthetic profile with a modulated fetal fraction (ff) compared to the synthetic profile of step c).

6. The method according to claim 1, wherein the value (E) is defined as:
$${E}_{ChrT} = \frac{ff * {n}_{( fetal \_ range )}^{chrT} - {n}_{( fetal \_ range )}^{genome} * ff * {p}_{chrT}}{\sqrt{{n}_{( fetal \_ range )}^{genome} * ff * {p}_{chrT} ( 1 - ff * {p}_{chrT} )}} ;$$
Where n_{(fetal_range)}^{genome} corresponds to the total number of reads with fragment length in a reference fetal range,
Where n_{(fetal_range)}^{chrT} corresponds to the number of reads with fragment length in the chromosome of interest (T) in a reference fetal range,
Where
$${p}_{chrT} = \frac{n reads chrT}{n reads genome}$$
corresponds to the number of reads on the chromosome of interest (T) divided by the total number of reads in the synthetic profile.

7. The method according to claim 6, wherein the chromosome of interest (T) selected from the group consisting of chromosomes 13, 18, 21, X and Y.

8. The method according to claim 7, wherein the chromosome of interest (T) selected from the group consisting of chromosomes 13, 18, and 21.

9. A method for determining the reliability of Non-Invasive Prenatal Testing (NIPT), using a decision tree trained beforehand on reference profiles, comprising the steps of:
a) providing, from a maternal biological sample, a fetal fraction (ff), a sequencing depth (sd), a synthetic profile and a value (E) corresponding to said synthetic profile, according to any of the preceding claims,
b) calculating a Z-score for said synthetic profile by comparing it to Z-scores of said reference profiles,
c) feeding said decision tree with said calculated Z-score and said fetal fraction, sequencing depth and value (E) of said synthetic profile, in order to classify said synthetic profile in a group by comparing said calculated Z-score to a Z-score threshold value, and
d) determining, from said classification, a reliability score (Rscore) for a NIPT of said maternal biological sample.

10. The method according to claim 9, wherein, at step c), said synthetic profile is classified in a group of aneuploid profiles or a group of euploid profiles, so as to determine the reliability of Non-Invasive Prenatal Testing in detecting fetal aneuploidy.

11. The method according to claim 10, wherein the fetal aneuploidy is a human fetal aneuploidy of a chromosome of interest (T) selected from the group consisting of chromosomes 13, 18, 21, X and Y.

12. The method according to claim 11, wherein the fetal aneuploidy is a human fetal aneuploidy of a chromosome of interest (T) selected from the group consisting of chromosomes 13, 18 and 21.

13. A device for implementing the method for assessing the fetal fraction (ff) and sequencing depth (sd) in Non-Invasive Prenatal Testing (NIPT) according to claim 1.

14. The device according to claim 13, comprising a decision tree trained beforehand on reference profiles, the device being configured to implement a method for determining the reliability of Non-Invasive Prenatal Testing (NIPT), comprising the steps of:
providing, from a maternal biological sample, a fetal fraction (ff), a sequencing depth (sd), a synthetic profile and a value (E) corresponding to said synthetic profile, according to any of the preceding claims,
calculating a Z-score for said synthetic profile by comparing it to Z-scores of said reference profiles,
feeding said decision tree with said calculated Z-score and said fetal fraction, sequencing depth and value (E) of said synthetic profile, in order to classify said synthetic profile in a group by comparing said calculated Z-score to a Z-score threshold value, and
determining, from said classification, a reliability score (Rscore) for a NIPT of said maternal biological sample.

15. A computer program product comprising a support and stored on this support instructions that can be read by a processor, these instructions being configured to assess the fetal fraction (ff) and sequencing depth (sd) in Non-Invasive Prenatal Testing (NIPT) according to claim 1.

16. A computer readable medium comprising the computer program product according to claim 15.

17. A computer program product comprising a support and stored on this support instructions that can be read by a processor, these instructions being configured to determine the reliability of Non-Invasive Prenatal Testing (NIPT) according to claim 9.

18. A computer readable medium comprising the computer program product according to claim 17.
